(19) United States
(12) Patent Application Publication
(10) Pub. No.: US 2026/0124064 A1
(43) Pub. Date: May 7, 2026

(54) DEVICE FOR PRODUCING AN EFFECT UPON A MALE REPRODUCTIVE SYSTEM OR PART THEREOF

(71) Applicant: Performance Industries AS, Oslo (NO)

(72) Inventor: Espen JENSEN, KRISTIANSAND (NO)

(73) Assignee: Performance Industries AS, Oslo (NO)

(21) Appl. No.: 19/117,538

(22) PCT Filed: Oct. 3, 2023
(86) PCT No.: PCT/NO2023/060060

§ 371 Date: Apr. 1, 2025

(30) Foreign Application Priority Data
Oct. 5, 2022 (NO) 20221063

(51) Int. Cl. A61F 5/41; A61H 19/00

(52) U.S. Cl. CPC A61F 5/41; A61H 19/32; A61F 2005/412; A61H 2201/0157; A61H 2201/1215; A61H 2201/123

(57) ABSTRACT

Devices for producing an effect on a male reproductive system or part thereof, and related apparatuses, are exemplified including a rigid cup for a penis head, coupling means for coupling the rigid cup to the penis with the penis head inserted in the cup, sealing means for sealing between the rigid cup and the penis when the penis head is inserted for preventing or hindering air from entering the cup, and a push/pull actuator configured to be coupled to the rigid cup and operable to move the cup with the inserted penis head reciprocally back and forth. A vacuum lock effect may lock the penis head to the cup upon push/pull movement. The device can be used for facilitating massage, erection, prolonged orgasm, or stimulation of the bulbospongiosus and/or pelvic floor muscles, or as a sex toy.

### CROSS-REFERENCE TO RELATED APPLICATIONS

This application is the U.S. national stage application of International Application No. PCT/NO 2023/060060, filed Oct. 3, 2023, which international application was published on Apr. 11, 2024, as WO 2024/076243 in the English language. The International Application claims priority to Norwegian Ser. No. 20221063, filed Oct. 5, 2022. The international application and Norwegian application are both incorporated herein by reference, in their entirety.

### FIELD

The present invention relates in particular to a device for producing at least one effect upon, e.g. stimulating or activating, a male reproductive system or part thereof, which device in use is applied to the penis.

### BACKGROUND

The male reproductive system is a complex system of parts, including the penis, erection muscles, glands, and related bodily tissue and material. Depending upon suitable stimulus, through communication with the brain and/or sensory inputs, such a system can perform to produce an erection of the penis, orgasm, ejaculation of sperm and sensations. For various reasons, the reproductive system may falter or may not function optimally or may not work as desired. Individuals may experience difficulties in obtaining an erection which may reduce or hinder sexual performance or pleasure. Large numbers of the world's male population experience erectile dysfunction to some extent during their lifetime. Erectile dysfunction can have various causes. However, an erection of the penis in humans depends upon proper functioning and activation of, the bulbospongiosus muscles (erection muscle) at the base of the penis, pelvic floor muscles, neurons, and good arterial blood circulation. If the various muscles and/or neurons involved do not function well, or if there is poor arterial blood circulation, difficulties obtaining an erection can ensue. Erectile dysfunction after surgery may for example require treatment that aims to improve, stimulate, or activate local arterial blood circulation, neurons, and the functioning of the muscles involved. Improving the erection of the penis can also be sought in individuals that do not necessarily experience dysfunction as such, but that seek for example a heightened or prolonged sexual experience.

Various solutions for facilitating erection function and sexual performance have been proposed and include for example medicaments such as Viagra®. Viagra® may be inefficient in the event of for example a neuron problem or depression, and may have side effects, and there can be large variation in the effect experienced by different users. Other solutions such as a penis ring and or a vacuum pump device may facilitate temporarily an artificial erection, but they may be inefficient for many medical recovery purposes, the inventors noting that these are based upon sustaining venous blood lacking in oxygen and nutrients statically in the penis. Many devices or solutions of prior art otherwise rely heavily upon the individual having well-functioning biological capacities for biological activation of the bulbospongiosus muscle (erection muscle).

Various devices are also known in prior art which an individual may operate to stimulate the penis. These devices of the prior art can produce mechanical vibrations which are imparted to the penis shaft or may comprise sleeves around the penis shaft that slide back and forth along the penis shaft. While devices of prior art can stimulate the penis locally to some extent for sexual pleasure or performance purposes, they are typically ineffective for medical purposes such as regaining full function of the different parts of the system. They may not produce much effect if any upon the physiological condition of the penis and the related muscles, recovery, arterial blood circulation and/or the longer-term erectile function of the individual.

There is a need for solutions for improving/boosting the function, recovery, and performance of male reproductive system. There is also a need for solutions that can provide enhanced sexual pleasure and prolonged orgasms. At least one aim of the invention is to obviate or at least mitigate one or more drawbacks of prior art.

### SUMMARY

According to a first aspect of the invention, there is provided a device for producing at least one effect on a male reproductive system or at least one part thereof, the device comprising: a rigid cup for a penis head; coupling means for coupling the rigid cup to the penis with the penis head inserted in the cup; sealing means for sealing between the rigid cup and the penis when the penis head is inserted for preventing or hindering air from entering the cup or entering a region between the cup and the penis head; and a push/pull actuator configured to be coupled to the rigid cup and operable to move the cup with the inserted penis head reciprocally back and forth.

Preferably, the rigid cup, the coupling means, and the sealing means are configured to together operate to obtain in use a vacuum lock effect between the rigid cup and the inserted penis head for substantially locking the penis head to the cup so that the penis head is forced to move along with the cup upon pull/push movement.

In use, the device can be used to reciprocate the penis head itself back and forth, and may transmit force, along the shaft of the penis, through the penis, and into bulbospongiosus muscle (erection muscle) and/or surrounding areas. The actuator can push against the inserted penis head in the direction along the penis shaft, toward the base of the penis shaft, and pull the inserted penis head in the opposite direction along the penis shaft, away from the base of the penis shaft, in accordance with the reciprocating movement of the actuator. In use, the penis head can thus be pushed by, and/or along with, the cup in one direction, e.g. a push direction, and the penis head can be pulled by, and/or along with, the cup in the opposite direction, e.g. a pull direction. The coupling means and/or sealing means can act to lock the penis head to the cup in use so as to be movable along with the cup and in so doing, it can be appreciated that the movement back and forth can take place without frictional slippage, gliding, or sliding of the cup with respect to the penis head.

The manner of such force transmission that may be obtained by the device may advantageously produce a dynamic mechanical activation and/or stimulation of the bulbospongiosus muscle (erection muscle) and surrounding area.

When pull forces are alternatingly imparted through the reciprocating movement to the penis head near the elastic stretch limit of the penis, the pull forces may act to further tension or stretch the penis along its shaft and/or may exert through the penis a further pull upon the bulbospongiosus muscle located at the base of the penis.

Various embodiments may be advantageous in that the device may, through imparting and transmitting push/pull forces through the penis, act as a pump for facilitating blood circulation and increasing arterial blood flow in the penis, bulbospongiosus muscle and surrounding area, where the arterial blood contains high level of oxygen and nutrients to fuel the muscle cells, neuron cell and surrounding area, for improving function, erection, recovery and performance of the male reproduction system.

Various embodiments may also be advantageous in that the device through transmission of force through the penis may produce and force dynamic contractions and expansions of the bulbospongiosus muscle allowing for male orgasm to last for several minutes.

Preferably, the rigid cup is configured to allow the penis head to be inserted and located against the material of the cup. The coupling means may preferably sealably couple the rigid cup to the penis. Thus, the penis head may be sealably inserted in the cup. Thus, air may be prevented or hindered from entering the cup and/or entering the region between the cup and the penis head. The penis head may be inserted and located against one or more surfaces of the base portion and/or walls of the cup. The cup or base portion thereof may have at least one base and/or wall surface against which an end surface of the inserted penis head may contact when inserted. The cup typically comprises containing walls extending from the base portion toward a penis insertion opening of the cup. The walls comprise typically closed walls so that air or fluid does not pass through the walls in use. The rigid cup is preferably configured to fit and be coupled to the penis head in sealed fashion without air entering into the cup.

The rigid cup may have at least one air or fluid escape channel through a wall section of the rigid cup for letting out air or fluid, e.g. air, gas, liquid or other fluid, from the cup when locating the penis head in the rigid cup. The channel through the wall can be provided with a valve member which may automatically allow air or fluid out of the cup upon insertion of the penis head and prevent air or fluid, from entering into the rigid cup through the channel when inserted. The valve member may be a one-way valve automatically operable to let out air or fluid through the channel in one direction but not in the other direction. Also, air or fluid which may accidentally enter the rigid cup in the region between the cup and the head of the penis may be evacuated via the air or fluid channel. In some variants, the rigid cup may be configured to connect to an evacuation device, e.g. a pump device, to evacuate fluid or air through the channel from the inside of the cup. This may be convenient if the air residing in the rigid cup may not be of sufficient pressure to pass through the channel and/or the valve unassisted. For example, if a self-opening or one-way valve is provided in the channel operable to open and let through air only when sufficient pressure is produced, then the evacuation device, e.g. the pump device, may be useful for assisting draw the fluid or air out of the cup and through the channel. The evacuation device, e.g. the pump device, may connect to an exterior of the rigid cup and may typically be hand operable. The pump device can comprise a squeezable bladder in communication with the channel, so that upon unsqueezing the bladder, flow from the channel toward the bladder is encouraged.

Advantageously, through locating the penis head against the base of the cup, typically with surface of the penis head in contact with surfaces of the wall and or base portion of the inside of the rigid cup and/or through the expulsion of air or fluid when the penis head is inserted, coupling of the rigid cup to the penis head may be facilitated, and/or push/pull motion may be imparted through the cup while mitigating potential slippage between the penis head and the cup upon operation, which can further facilitate the transfer of push-pull motion to the penis and the effect of the push-pull actuator upon the penis toward obtaining an erection.

In particular, the coupling means and sealing means are configured to provide coupling to the penis head when inserted and sealing between the rigid cup and the penis for preventing or hindering air or fluid ingress. In this way, the rigid cup can in effect be substantially locked to the head of the penis, such that when the penis head is pulled by the rigid cup, e.g. when the actuator moves the cup or the device moves and pulls on the penis away from the user's body, the penis head tissue is pulled with the cup and moves along with it. A vacuum effect may be produced in the rigid cup between the penis head and the cup itself, so as to help to substantially lock the penis head to the rigid cup so that the penis head travels back and forth along with the cup, in use. Upon pulling, the vacuum effect may keep the penis head locked to the rigid cup. The substantially locking of the penis head to the cup so provided is termed herein a “vacuum lock”.

Typically, the cup is disposed on a drive part. The drive part may be configured to extend between the rigid cup and a drive mechanism of the actuator at a location e.g. beyond the penis head in a direction away from the base of the penis shaft. The drive part may typically comprise a connecting stem between the receptacle and the actuator. The drive part is preferably movable linearly back and forth by the actuator.

Typically, the drive part comprises an elongate stem comprising a longitudinal axis configured to be arranged coaxially with a shaft axis of the penis, in use.

Typically, the rigid cup and the actuator are configured to be detachable from one another. For example, the rigid cup can be detachably connectable to the actuator. Advantageously, the rigid cup can be disconnected and separated from the actuator. A rigid cup of one size may be removed and be replaced with a rigid cup of another size. The rigid cup may be removed so that it can be more conveniently cleaned. The detached rigid cup may first be located upon the penis head and coupled to the penis using the coupling means, and thereafter, once the cup has been fitted, the actuator may be attached to the rigid cup and operated. This can be convenient and facilitate ease of coupling the cup to the penis. The rigid cup may also be configured to be detachably coupled to the actuator so that when a certain load safety limit is reached, e.g. load applied in the axial direction upon pulling the actuator away from the rigid cup, the actuator detaches. In this way, injury from excess loading may be preventable.

In variants of the drive part comprising a stem, the stem may have a fitting for releasable connection or disconnection of the rigid cup from the stem. The stem may comprise a fitting for releasable connection or disconnection of the rigid cup. Typically, the coupling means is arranged to extend around the penis, preferably around the shaft at or near the penis head. The coupling means may comprise a sleeve or ring. The sleeve may be or comprise a flexible membrane configured to extend around the penis when inserted. The flexible membrane may be resilient and/or elastic and may conform to and grip the penis shaft. The coupling means may secure the inserted penis in location in the rigid cup. Typically, the coupling means is configured to grip the shaft of the penis at or near the penis head.

Typically, the sleeve is arranged to seal between the penis and the receptacle for hindering or preventing air from entering into the cup when the penis head is inserted.

Advantageously, the coupling means can be arranged to be coupled to the penis with the penis fully inserted into the rigid cup and located against the cup, e.g. against the base of the cup, for securing the penis in fully inserted location in the cup, and for avoiding or mitigating frictional slippage of the rigid cup relative to the penis head and/or shaft under reciprocation of the rigid cup.

Typically, the coupling means is movable between a first configuration in which the coupling means is supported on the rigid cup for permitting insertion of the penis head into the receptacle and a second configuration for coupling the cup to the penis. For example, the coupling means may comprise a sleeve which is unrollable to move the coupling means from the first configuration to the second configuration, wherein in the second configuration the unrolled sleeve is configured to grip the penis.

Typically, the actuator comprises a drive mechanism. Optionally, the actuator comprises an electrical solenoid-actuator. The rigid cup may in such examples be coupled to the electrical solenoid-actuator, which can thus operate to move the rigid cup reciprocally back and forth. The electrical solenoid-actuator comprise a reciprocally movable drive part. The rigid cup and/or stem of the rigid cup may be connected to the drive part and/or solenoid actuator.

Typically, the device further comprises a hand-held casing from which the reciprocally movable drive part extends. In variants where the actuator comprises a drive mechanism, the drive mechanism is typically contained within the casing. In other variants, the electrical solenoid-actuator may be contained within the casing.

Typically, the device further comprises control means for operating the actuator, and in variants using the drive mechanism, controlling the drive mechanism. The control means may comprise one or more user controllable buttons or switches or other devices.

Optionally, the actuator or the drive mechanism is operable and/or controllable under remote control. Such remote control may be provided through any one or more of: a network; WIFI; computer device; mobile phone device; computer program.

Optionally, the device can have a strap attached thereto, which strap can be configured to extend around a neck of the user to support the device in position when in use. This may facilitate use of the device by users with disablements not able to hold the device effectively during use.

Typically, the control means are operable to vary either or both the rate of reciprocation and the amount of extension of the drive part from the casing in the cycle of travel of the drive part.

In certain embodiments, the actuator may operate to move the cup reciprocally back and forth with fixed travel length or range of travel. In other embodiments, the actuator may operate to move the cup reciprocally with travel length or range of travel that can be varied and preferably adjusted. In this way, the travel length or range of travel may be altered or adjusted to obtain to a desired travel length or range of travel.

Typically, in embodiments where the actuator comprises a drive mechanism and is operable to reciprocate the cup with fixed length or range of travel back and forth, the drive mechanism is configured for fixed length or range operation, and may comprise: a motor; a rotary mover arranged to be driven and rotated by the motor; a linear drive part arranged to be movable linearly back and forth; and a coupling member arranged to extend between the rotary mover and the linear drive part, the coupling member having a first end which is coupled to the rotary mover and a second end which is coupled to the linear drive part.

Typically, in embodiments where the actuator is operable for varying the length or range of travel of the reciprocating cup, the drive mechanism is configured for varying and allowing adjustment of travel length or range of travel, and may comprise: a motor; a rotary mover arranged to be driven and rotated by the motor; a linear drive part arranged to be movable linearly back and forth; an adjustable support member; and a link member supported upon the adjustable support member via a rotational coupling; a first coupling member arranged to extend between the link member and the rotary mover, the first coupling member having a first end which is coupled to the rotary mover and a second end which is coupled to the link member; and a second coupling member arranged to extend between the link member and the linear mover, the second coupling member having a first end which is coupled to the link member and a second end which is coupled to the linear drive part; wherein the support member is positionally adjustable to move the rotational coupling between a first location and a second location, for varying the range of travel obtainable by the linear drive part when operating the rotary mover.

Typically the drive mechanism with adjustable travel length or range further comprises a gear arrangement operable to urge the adjustable support member from one position to another.

Typically the drive mechanism with adjustable travel length or range further comprises an adjuster for adjusting the position of the support member and control means for operating the adjuster, the control means being operable by a user by means of one or more user-operable control devices.

Producing at least one effect upon a male reproductive system or at least one part thereof, may be or comprise any of: stimulating a male reproductive system or at least one part thereof; or facilitating in obtaining an erection or improving an erection function of the penis. The device in any embodiment may be termed a reciprocator device.

The device may include any one or more further features as described anywhere else herein or as described in relation to any other aspect of the invention wherever described herein.

According to a second aspect of the invention, there is provided apparatus for determining the extent of erection of a penis, the apparatus comprising: a device in accordance with the first aspect of the invention, and further including a motor for operating the actuator and means for detecting an operational property of the motor; and a determiner configured to use the detected operational property to determine the extent of erection of the penis.

Embodiments of the invention can be advantageous in various ways as apparent from the specification throughout.

### BRIEF DESCRIPTION OF THE DRAWINGS

There will now be described, by way of example only, embodiments of the invention, with reference to the accompanying drawings, in which:

FIG. 1 is a side-sectional representation of a device for stimulating a male reproductive system, shown coupled to a head of the penis, according to an embodiment of the invention;

FIG. 2 is a side view representation of the device of FIG. 1;

FIG. 3 is a side view part-sectional and simplified anatomical representation showing the device of FIGS. 1 and 2 in use on a penis of a person;

FIG. 4 is a perspective view of a configurable drive mechanism for use in the device of FIGS. 1 and 2;

FIG. 5 is a side view of the drive mechanism of FIG. 4;

FIGS. 6A & 6B are side view representations of the drive mechanism of FIGS. 4 and 5 at respective end positions of the linear travel of the drive part in a first configuration of the drive mechanism, travel length adjusted for greater range of travel and extension from the actuator;

FIGS. 7A & 7B are side view representations of the drive mechanism of FIGS. 4 and 5 at respective end positions of the linear travel of the drive part in a second configuration of the drive mechanism adjusted for shorter range of travel and extension from the actuator;

FIG. 8 is a perspective view of the drive mechanism from a different angle;

FIG. 9 is a perspective representation of the drive mechanism from a different angle; and

FIG. 10 is a top view representation of the drive mechanism of with an arrangement of batteries on top.

### DETAILED DESCRIPTION OF THE DRAWINGS

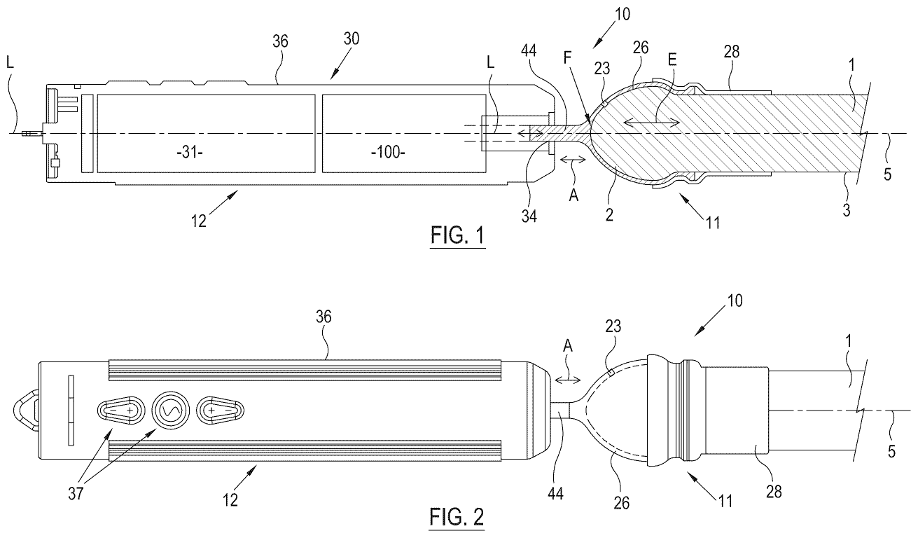

With reference to FIGS. 1 and 2, a device 10 for producing at least one effect upon, e.g. stimulating, a male reproductive system is depicted, in use, applied to a penis 1. The device 10 has generally two parts, one part 11 which is arranged to be affixed to a head 2 of the penis 1, and another part 12 which comprises an actuator 30 to effect push/pull reciprocating movement of the part 11 affixed to the penis head, relative to the other part 12, as indicated by arrows A. Thus, the device 10 may be regarded a reciprocator device.

The part 11 connecting to the penis head 2 comprises a rigid end cup 26 into which the penis head 2 is received, and a coupling means in the form of a sleeve 28 for attaching and securing the rigid end cup 26 to the penis 1. The rigid end cup 26 extends around and over the penis head end.

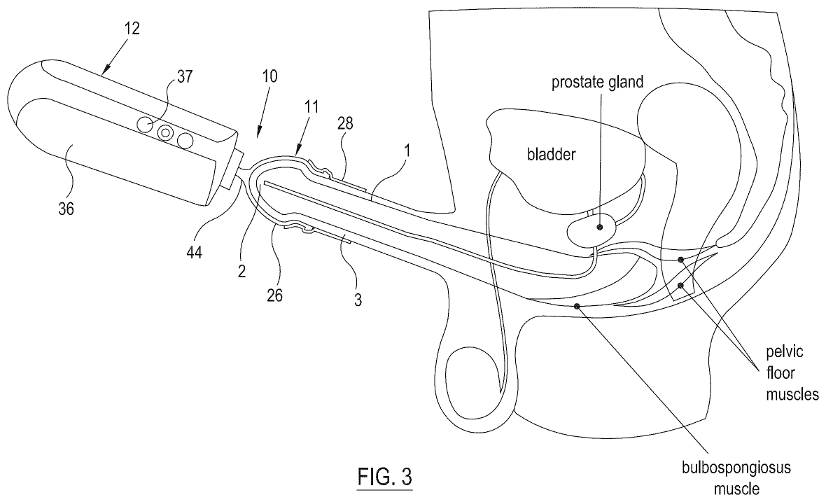

The sleeve 28 extends from the end cup 26 along the shaft axis 5 toward the base of the penis shaft. The sleeve 28 extends around, contacts and grips the penis shaft at or near the penis head 2, and in this way holds the penis head in position within the end cup 26 and keeps the end cup 26 connected to the head of the penis. The penis 1 is arranged with the tissue of the penis head 2 exposed and located in the end cup 26, typically in use in contact with the base end and surrounding surfaces of the inside of the end cup 26. In this configuration, therefore, the device 10 is connected to the penis head 2, and the reciprocating movement of the end cup 26 as indicated by arrows A, when actuated, is translated to impart a corresponding movement to the penis head 2 itself. Thus, when the device 10 is operated by reciprocating movement of the end cup 26, the penis head 2 is manipulated with a corresponding push/pull movement being imparted to the penis head 2. The push/pull of the penis head 2 is therefore produced relative to the actuator part 12 which is typically held and supported in position by a user. So, the device can be applied in use so that as the end cup 26 in FIG. 1 moves toward the right, the penis head is pushed toward the right, and as the end cup 26 moves left, the penis head is pulled toward the left. Using the device 10, the penis is moved, i.e. pushed and pulled back and forth preferably substantially without frictional sliding between the penis and the end cup 26 or sleeve 28. The push/pull of the penis head 2 initiated from the reciprocating end cup 26 facilities to transmit force through the penis along the penis shaft 3 toward the base of the penis 1 and further into the bulbospongiosus muscle, pelvic floor muscles and surrounding area. The pull upon the penis 1 repeatedly within the elastic stretch limits of the penis provides an effect felt by the pelvic and erection muscles even when the penis is otherwise bendable. Transmission of force through the penis tissue in this manner can facilitate stimulating blood flow and activating the erection muscles in the pelvis, which may facilitate obtaining an erection, as indicated in FIG. 3. This may be for pleasure or therapy purposes. In treatment scenarios, the device 10 may be used for facilitating to increase arterial blood flow with high level of oxygen and nutrients/fuel in the bulbospongiosus muscle and surrounding area facilitating recovery of muscle cells, neurons and surrounding tissue along with activation, stimulation, training or retraining the erection muscle, for example. Arterial blood circulation can prevent deterioration of neurons and muscle cells in the relevant area during a recovery process with otherwise low level of arterial blood flow.

The sleeve 28 is arranged to seal around the penis shaft 3 to prevent air from entering into the end cup 26. This is because it is sought to avoid air entering into the cup between the penis head and the cup. Such sealing can help to maintain the connection between the end cup 26 and the penis head 2, so that force can be effectively transmitted from the reciprocating end cup 26 into the tissue of the penis head 2 and along the penis shaft 3. Similarly, it is sought that the penis head is fully inserted to the end of the cup 26, and that the cup 26 forms a contact fit to the penis head 2, as depicted in FIG. 1. In this example, the end cup 26 also has an air outlet 23 for letting air out of the cup 26 upon insertion of the penis into the cup 26. The air outlet 23 comprises a passageway through the walling of the cup 26, the passageway comprising a valve which generally is closed but which is openable automatically by pressure differential to let air out of the cup through the passageway to surroundings. Allowing the air to be bled out of the cup can facilitate insertion of the penis into the end cup 26 and facilitate providing suitable contact and coupling of the cup 26 to the penis head 2. Indeed, the sealing and coupling to the penis is typically such that a vacuum lock effect may be produced upon pull movement, between the cup and the penis, so that the end cup 26 is substantially locked to the penis head 2 and the penis head 2 is moved with the end cup 26 back and forth, as indicated by arrow E, without friction or sliding between the cup and the penis head. The term “locked” herein can be understood to indicate that tissue of the penis head is kept substantially in fixed position with respect to the end cup. It is generally sought to avoid the presence of air in the region indicated by arrow F between the end cup 26 and the penis head 2, because the air can be expected to compress and decompress reducing the sharpness in transition of forces when alternatingly changing direction from pull to push or vice versa during reciprocation. Basically, air between can function as a shock absorber resulting in high force transfer losses, forces may act more locally on the penis head, and may not propagate deeply into inner area of the male genitalia or reproductive system.

The second part 12 of the device 10 comprises in this example an actuator 30 for operating the device 10 and producing the reciprocating movement of the end cup 26 in use. The actuator 30 includes a drive mechanism 100 through which the rotary motion of an electric motor is transmitted and transformed into a linear reciprocating motion of a drive part. The drive mechanism 100 is housed within a housing 36. Various electronics 31 and control means for operating the drive mechanism 100 are also housed in the cylindrical housing 36. Hand operable control buttons 37 are in this example provided on the housing 36 for the user to adjust the rate and extent of travel of the reciprocation of the drive part.

The end cup 26 is provided on a stem 44 by which the end cup 26 is coupled to the actuator 30. The stem 44 (which constitutes a drive part movable by the drive mechanism) extends from an end of the cylindrical housing 36. The stem 44 is further located in a channel 34 in the housing and is arranged to be movable along the channel 34 relative to the housing 36. The actuator 30 includes the drive mechanism 100 which is operable by a motor to move the stem 44 (and consequently the end cup 26) reciprocally and linearly back and forth with respect to the housing 36 of the second part 12, as indicated by the arrows A.

The housing 36 in this example is configured to be hand-held and hand operable, by the housing 36 having switches and/or buttons 37 for varying the rate of reciprocation and/or the varying the distance of travel of the stem 44 or end cup 26 between end points in the movement cycle. For example, at one setting, the stem 44 may travel 15 mm in each direction back and forth, and at another setting, the stem 44 may travel 5 mm in each direction back and forth. The speed of reciprocation is also adjustable. At one setting, the cycle of travel back and forth is shorter or longer than at another setting, adjustable according to requirements. The travel range or length can also be adjusted to have any value within the range of 5 to 15 mm. The device 10 can in other examples be configured to provide adjustably variable travel length over different ranges of values, for example with either or both the upper and lower end points within the range of 5 to 15 mm.

The amount of travel of the stem part 44 in each stroke, i.e. travel length or range of travel, can be varied and adjusted according to needs. The rate of reciprocation, i.e. stroke rate, may also be varied conveniently to further customise the behaviour of the device. The range of travel and rate of reciprocation can be varied smoothly without stopping the action of the actuator 30. The rate can be set anywhere in the range of up to 3000 rpm. To this end, the drive mechanism 100 is configured and adjustable as described in further detail below.

As can be appreciated, the stem 44 extends away from the end cup 26 at the penis head toward the actuator housing 36. The stem 44 is arranged to be moved axially, preferably coaxially with and along the longitudinal axis 5 of the penis shaft 3. The actuator 36 is configured to be arranged beyond the penis head end and with the end of the housing 36 from which the stem 44 protrudes arranged opposite the penis head 2.

In more detail still, the sleeve 28 in this example comprises an elastomeric material. The sleeve 28 is stretchable elastically and adaptable to fit around the penis shaft 3 at or near the penis head 2, and grip and form the seal around the penis 1. In various examples, the sleeve 28 is a stretchable tubular membrane. The one end of the sleeve 28 is fitted securely to the end cup 26 and extends around a lip at an entrance end of the cup. For easy coupling when the device 10 is to be used, the sleeve 28 may be rolled back or otherwise provided in a stowed configuration on the cup structure and then after insertion of the penis head into the end cup 26, the sleeve 28 may be unrolled or otherwise arranged along the shaft 3 to engage and connect with the penis shaft 3 to obtain a connecting configuration for operation. The sleeve 28 in other variants can be of different materials.

The end cup 26 in this example is formed of a plastics material, although in other variants, other suitable materials may be used. In certain variants, the end cup 26 is lined with a liner or a liner is used between tissue of the penis head and a wall portion of the end cup 26, which liner is for example removably inserted into the end cup and/or is pre-applied to the penis head before insertion.

In some variants, the sleeve 28 can be omitted and other means of coupling the actuator and/or receptacle to the penis head can be provided. For example, part of the walling of the end cup 26 may be flexible and a band, tape, clamp or other means may be provided on the end cup 26 so that the flexible part of the walling of the end cup grips onto the penis 1. In another example, the walling of the end cup 26 could have sealing flexible members on an inside of the walling of the end cup. Many other variants are possible.

Furthermore, although described as a rigid end cup 26 in certain examples as mentioned above, other rigid cups may be employed similarly to receive and provide suitable coupling of the actuator to the penis head 2, whereby the penis head 2 can be manipulated similarly, e.g. gripped and moved in push/pull and substantially friction free manner in correspondence with movement of the actuator.

In some variants, the end cup 26 is detachably connected to the actuator 30. In one such example the end cup 26 is detached from the stem 44. In another, the stem 44 with the end cup 26 attached is detachable from part of the actuator mechanism.

By providing a detachable end cup 26, a range of end cups 26 and/or sleeves 28 of different sizes can be provided, and the user can select one that is suitable and connect that to the actuator 30. The end cups 26 can be provided with the sleeves 28 pre-attached. A detachable end cup 26 can allow for easy cleaning and easier to connect/couple to the penis and disconnect.

In the above, the housing 36 is a hand-held casing. The housing 36 can in other embodiments have a connection for a strap (not shown) which may be worn around a neck of a user to support the device in location on the penis head during use, which may have advantages in allowing use of the device for users with disabilities.

By use of the device 10, improved function of the reproductive system may be obtained. By use of the device 10, the effects produced upon the reproductive system or part thereof can include any one or more of the following: improving performance of the male reproductive system or part thereof; stimulating the male reproductive system or part thereof; providing stimulation, sensation, activation, and/or obtaining improved performance of the male reproduction system; creating function, sensations and/or performance of the male reproduction system, including for example stimulating neurons and obtaining dynamic activation of the erection muscle and surrounding structure, and increasing arterial blood circulation. The device 10 can typically be operable to produce any of the following effect upon the reproductive system or parts thereof such as the penis and related muscles and tissue: stimulating or activating blood circulation; stimulating or activating nerves; stimulating, activating, exercising muscles and/or other tissue; and/or activating or stimulating male reproductive muscles and/or part. Since the friction free coupling of the rigid cup 26 to the head 2 of the penis 1 is provided for, a more direct transmission of reciprocating force can be obtained from the actuator and directed in the longitudinal direction of the penis 1. As such, the movement produced by the reciprocator device and coupling to the penis head can facilitate to transmit force deep into structure of the penis and can produce a dynamic activation of the erection muscle/bulbospongiosus muscle at the inner root of the penis. The device 10 may in this way act to mechanically to increase arterial blood circulation (akin to a pump) and provide stimulation and or activation of neurons, the penis head, the penis, pelvic floor muscles and surrounding areas. Erection may be facilitated and erection functionality of the penis may be improved.

In various embodiments, advantageously the device can facilitate in obtaining an erection of the penis, prolonged orgasm, increased arterial blood circulation, creating forces propagating deeper through the whole penis enforcing dynamic activation and stimulation of the erection muscle (bulbospongiosus), neurons, muscle cells and surrounding area, facilitate improved recovery from surgeries in the male genitalia or create a different sensation.

The device 10 provides for movement of the penis head reciprocally back and forth along with the rigid end cup 26 to which it is coupled, which as mentioned can facilitate providing an effective transmission of force through the penis. This can be contrasted significantly to prior art devices designed for frictional sliding and gliding of parts along the penis or penis head, the sliding and gliding resulting in significant force transmission loss along the penis. Prior art devices with soft, flexible, or elastic material over the penis head can similarly suffer sliding/gliding effect force losses along the penis shaft.

The actuator 30 comprises a drive mechanism 100 which will now be described in further detail as example means for producing reciprocating movement of the stem 44 and in turn the end cup 26. In this example the drive mechanism 100 is configured to vary the travel length or range of travel of the reciprocation.

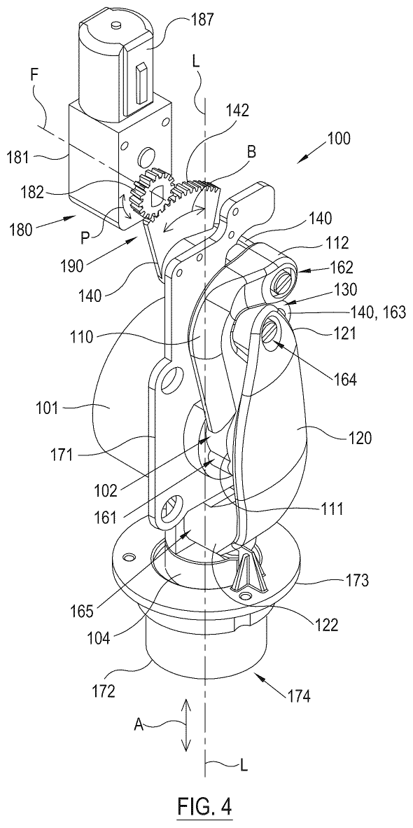

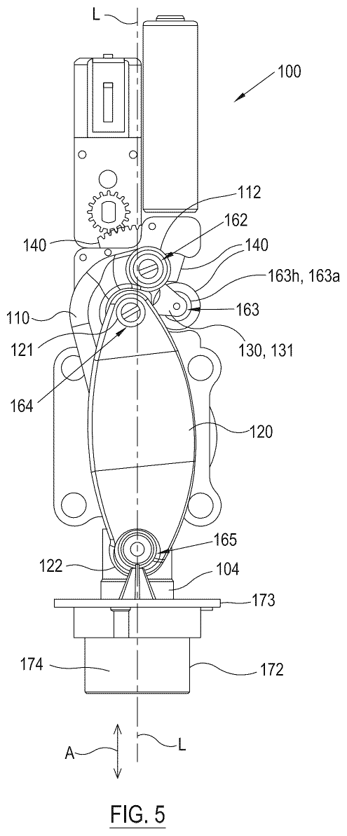

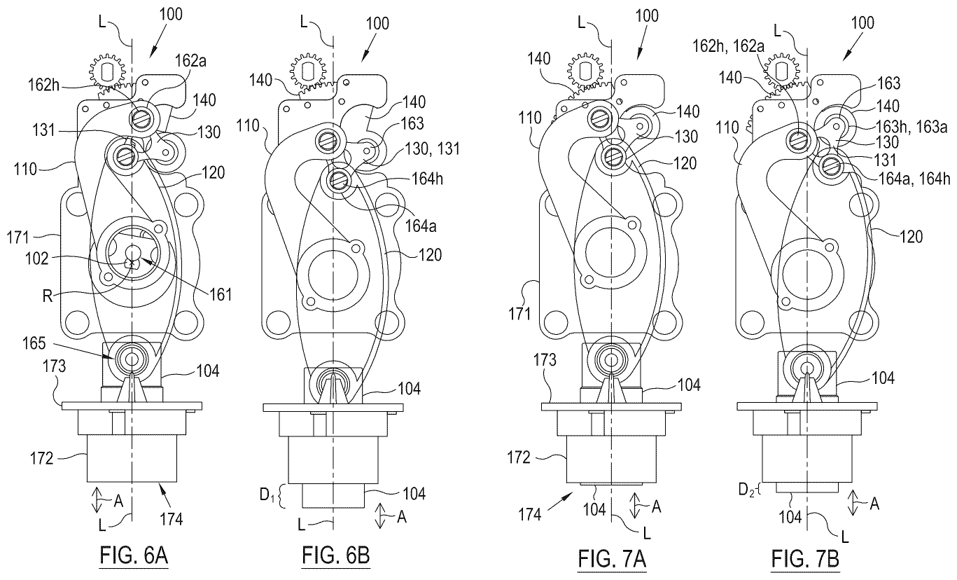

Turning first to FIGS. 4 and 5, a configurable drive mechanism is generally depicted at reference numeral 100. The drive mechanism 100 is configured to produce linear reciprocation of a drive part 104. The drive part 104 is arranged to be movable back and forth in the direction indicated by the arrow A. The drive mechanism 100 has an electric motor 101 which is operable to drive and rotate a rotary mover in the form of rotary shaft 102 (see also FIG. 6A). Further, the drive mechanism 100 includes a first coupling member 110, a second coupling member 120, a link member 130, and an adjustable support member 140.

The first coupling member 110 extends between the rotary driver 102 and the link member 130. The first coupling member 110 has a first end 111 which is coupled to the rotary driver 102, at first coupling 161, and a second end 112 which is coupled to the link member 130, at a second, rotational coupling 162.

The link member 130 is coupled to the adjustable support member 140 at a third, rotational coupling 163.

The second coupling member 120 extends between the link member 130 and the linear drive part 104. The second coupling 120 member has a first end 121 which is coupled to the link member 103, at a fourth, rotational coupling 164, and a second end 122 which is coupled to the linear drive part 104, at a fifth, rotational coupling 165.

The second to fifth rotational couplings 161-165 are rotatable about respective axes perpendicular to the longitudinal axis L and the direction A of linear reciprocation of the linear drive part 104. The axes of rotation of the second to fifth rotational couplings 161-165 are parallel axes.

The adjustable support member 140 is positionally adjustable and movable in an arc as indicated by arrow B. The movable support member 140 is mounted on a base plate 171 which in turn can be fixed to a body of a product. The movable support member 140 is movably mounted to the base plate 171, and thus is movable relative to the base plate 171 as indicated by arrow B. Also conveniently in this example, the motor 101 is mounted to the base plate 171. The base plate 171 in turn can be affixed to the housing 34 of the part 12 of the device 10.

As the adjustable support member 140 is moved along the arc, so too the third coupling 163 (between the support member 140 and the link member 130) is moved. In this way, the range of travel obtainable by the linear drive part 104 in response to operation of the rotary driver 102 is variable. The third rotational coupling 163 in its entirety is moved, i.e. travels, along with the support member 140 between a first location and a second location relative to the base plate 171. The third rotational coupling 163 upon movement into a different location supports the first and second coupling members 110, 120 at different distances away from the rotary driver 102. The range of travel obtainable by the linear drive part 104 in response to operation of the rotary driver 102 varies depending upon the position of the support member 140 and the movement of the support member 140 from one position to another, whereby the rotational coupling 163 is moved to another location.

The drive part 104 is located in a channel 174 and is arranged to move slidably back and forth along the channel 174. The channel 174 extends longitudinally along axis L. The channel 174 provides support for the drive part 104 laterally and helps to guide the moving drive part 104. The channel 174 in this example extends through a structure 172 comprising a collar 173 which can facilitate its fixing to a body of a product (not shown).

With reference now additionally to FIGS. 6A and 6B and FIGS. 7A and 7B, the travel of the drive part 104 in different configurations of the drive mechanism 100 can be further appreciated. In FIGS. 6A and 6B, the adjustable support member 140 is in a first position, and in FIGS. 7A and 7B the adjustable support member 140 is in a second position. In the second position, the third coupling 163, that is the coupling of the link member 130 to the support member 140, is moved away from the first location to a second location further along the axis L, so that the range of motion transferable through the link member 130 from the first coupling member 110 to the second coupling member 120 along the axis L is reduced. The drive part 104 is movable reciprocally back and forth between first and second end positions of each cycle. FIGS. 6A and 7A show the configuration of the drive mechanism 100 at the first end position within a cycle, and FIGS. 6B and 7B show the configuration of the drive mechanism 100 at the second end position within the cycle. When the adjustable support member 140 is in the second position as seen in FIG. 7B, the link member 130 between the first and second coupling members 110, 120 obtains a greater component extent in the direction transverse to the axis L than when the support member 140 is in first position as seen in FIG. 6B.

In one cycle back and forth, the drive part 104 is moved between a first end position and a second end position, and the distance of travel from the first to the second end positions per cycle with the support member 140 in the first position (FIG. 6A) is greater than that when the support member 140 is in the second position (FIG. 7A). The extent of the travel and the amount that the drive part 104 extends from the channel 174 can be varied by moving the support member 140 between the first and second positions. As can be seen in FIG. 6B, the drive part 104 travels and extends from the end of the channel 174 by the distance D₁, whereas in FIG. 7B the drive part 104 travels and extends from the end of the channel 174 by the distance D₂. The distance D₂ is significantly less than D1.

The support member 140 can be adjustably moved into different positions, and in this example the drive mechanism 100 includes an adjuster 180 for adjustably moving the adjustable support member 140 into different positions. The adjuster 180 is arranged to engage the support member 140 through a gear arrangement 190. More specifically in the example depicted, the adjuster 180 comprises a servo actuator 181 which drives a pinion gear 182 which in turn engages an arcuate tooth track 142 on the support member 140. The servo actuator 181 is operable to rotate the pinion gear about a fixed axis F. The servo actuator 181 is configured to be mounted in fixed position to a body of a product (not shown). The pinion gear 182 engages the tooth track 142. Upon rotating the pinion 182 on the tooth track 142, the support member 140 is urged from one position to another, relocating the coupling 163 of the link member 130 to the support member 140.

In other variants, the support member 140 is moved between positions in other ways, for example using other means than gears for engaging and/or moving the support member 140. Other adjusters can be employed to the same end of moving the position of the adjustable support member 140 and coupling point 163. The gear arrangement 190 can also take other forms.

The servo actuator 181 in this example is motorized and includes a motor 187 which is electrical. The electrical motor 187 is controllable, which includes that the motor speed is controllable and/or activating the motor. The electrical motor 187 in some variants is controllable by user controls, e.g. by buttons, switches, or the like. The electrical motor 187 in some variants is operable in accordance with pre-programmed instructions. The motor 187 in some variants is operable by remote control. By activating the motor 187, the adjuster 180 operates to adjust the support member 140 to vary the extent of linear travel of the drive part 104.

The drive motor 101 for producing the reciprocation of the drive part 104 is also electric and is also controllable to operate at different speeds. The drive motor 101 is controllable by user controls, e.g. by buttons, switches or the like. The electrical motor 101 in some variants is operable in accordance with pre-programmed instructions. The drive motor 101 in some variants is operable by remote control. A higher speed of the drive motor 101 increases the rate of linear reciprocation of the drive part 104.

The first coupling, between the first end 111 of the first coupling member 110 and the rotary mover 102 is fixed coupling, where the first end 111 is arranged eccentrically with respect to the axis of rotation R (see FIG. 6A) of the rotary mover 102. The first end 111 orbits therefore around axis R and the coupling member 110 has a component of movement along the long axis L. Furthermore, the arm extends between the first end 111 and the second end 112 on curved “swan-neck” trajectory. This facilitates coupling to the link member 130 beyond the first end 121 of the second coupling member 120. The second end 112 upon rotation of the rotary movement with a transverse component of movement which is accommodated by the rotational coupling 163 of the link member 130 to the support and the rotational coupling 162 of the second end 112 of the first coupling member 120 to the link member 130.

The link member 130 in the example comprises a triangular plate 131 with the second, third and fourth rotational couplings 162, 163, 164 defining three corners of a triangle of the plate 131. The link member 130 has coupling hubs 162h, 164h on the plate 131 which are arranged in a complementary coupling apertures 162a, 164a in the first and second coupling members 110, 120 respectively. The link member also has coupling hub 163h on the reverse side arranged in a complementary coupling aperture 163a in the support member. The coupling hubs 162a, 163a, 164a are supported in the complementary coupling apertures 162a, 163a, 164a on bearings, e.g. roller or ball bearings. These rotational couplings 162, 163, 164 can therefore provide low friction couplings which allow rotation between parts relatively freely and facilitate which can reduce wear. In other variants, the converse coupling arrangement is possible with the coupling hubs 162h, 164h, 163h provided on the coupling members 110, 120 and support member 140 and the coupling apertures 162a, 163a, 164a provided in the link member 130.

The fifth coupling 165 of rotary type also has a coupling hub on either the drive part 104 or the second end 122 of the second coupling member 120 and a complementary coupling aperture on the other of the drive part 104 and the second end 122, supported in bearing relationship with bearings therebetween, e.g. roller or ball bearings. The couplings 164 and 165 accommodate together with the rotational coupling 163 components of movement imparted from the first coupling member upon rotation of the rotary mover 102 in dependence upon the location of the support member 140.

In use, a user may switch on the drive motor 101. The drive motor 101 then rotates the rotary mover 102 at a certain speed. The first coupling member 110 follows the rotation and at the coupling 162 to the link member 130 moves relative to the support member 140, with the movement accommodated by rotation of the link member 130 at the coupling 163 relative to the support member 140, rotation at the couplings 162, 164 between the link member 130 and the first and second coupling members 110, 120, and rotation at the coupling 165 between the second coupling member 120 and the drive part 104 which is constrained in the channel 174 to move in the in the defined linear direction A. A certain extent and distance of travel of the drive part 104 is obtained per cycle back and forth linearly as the rotary mover 102 rotates. The rotary part 102 can then be applied to impart reciprocating movement to other components or structures depending upon the product. The speed is optionally varied to change the rate or reciprocation. The adjuster 180 is optionally operated and controlled to move the position of the support member 140 and the location of the coupling 163 between the link member 130 and the support member 140, and thereby vary the rate of reciprocation, which is possible without requiring to first stop the motor 101, i.e. it can be done while the drive part 104 is moving reciprocally back and forth, and can be done at an instant and at the option of the user without requiring dismantling or intervention with tools, and whilst the operational configuration of the drive mechanism is maintained. The amount of extension of the drive part 104 can therefore readily be increased or decreased as required.

The drive mechanism 100 can be incorporated into the body of a product for example into the handle or other part of the body of a hand-held product, e.g. the casing 36 of device 10. The casing 36 is configured to be gripped in one hand of a person and activated and adjusted as to reciprocation travel length and rate by the person gripping around the part 12 and for example using the buttons 37. The coupling members 110 and 120 of the drive mechanism 100 are arranged side by side in overlap and both extend generally between their first and second ends along the longitudinal axis L. This can facilitate a compact arrangement within the casing 36.

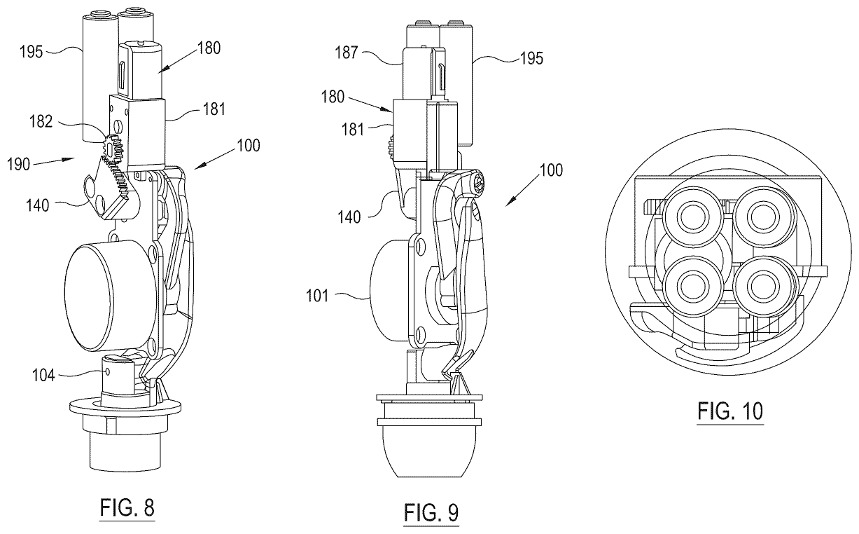

In FIGS. 8, 9 and 10, the arrangement of the drive mechanism 100 with batteries 195 for operating the motors 101, 187 and the adjuster 180 is illustrated. The compact nature can be seen. The drive mechanism 100, adjuster 180, and batteries 195 are fitted within a limited diameter as indicated in FIG. 10 to fit inside the casing 36, yet a robust and reliable reciprocating mechanism can be provided that is configurable to vary the obtainable extent of travel for the linear drive part 104.

The devices 10 described herein can be conveniently used also to determine the status and extent of erection of the penis. The inventors have noted that the reciprocation of the rigid cup 26 using the actuator 30 of an erect penis 1 provides greater resistance than when the extent of erection is less. The drive motor 101 may thus use more current when erection extent is greater and vice versa. Thus, there is also proposed apparatus for determining the extent of erection of a penis 1, the apparatus comprising: a device 10 described above with a drive motor 101 for operating the actuator 10 and means for detecting an operational property of the motor 101, and a determiner configured to use the detected operational property to determine the extent of erection of the penis 1. The property may be the current or other electrical property or motor status property which corresponds to the resistance experienced by the motor 101 as erection extent varies. This may be measured by suitable available sensors for monitoring or measuring the current supplied to the motor. Using the detected status, in a determiner, e.g. a computer device, one may determine the extent of the erection, and monitor this over time, upon use of the device 10.

Various modifications and improvements may be made without departing from the scope of the invention herein described. Although the actuator 30 can have a drive mechanism 100 as set out above for producing a linear reciprocating movement back and forth of the drive part 104, the actuator 30 can take many forms, and in one alternative for example comprises a solenoid actuator. The solenoid actuator in various examples has a first part comprising an solenoidal coil and a second part arranged in proximity to the second part and comprising a magnet, such that in dependence upon an electrical signal transmitted through the coil, the second part is caused to move back and forth.

1.-26. (canceled)

27. A device for producing at least one effect on a male reproductive system or at least one part thereof, the device comprising:
a rigid cup for a penis head of a penis;
coupling means for coupling the rigid cup to the penis with the penis head inserted in the cup;
sealing means for sealing between the rigid cup and the penis when the penis head is inserted for preventing or hindering air from entering the cup or entering a region between the cup and the penis head; and
a push/pull actuator configured to be coupled to the rigid cup and operable to move the cup together with the inserted penis head reciprocally back and forth;
the coupling means and the sealing means being configured to obtain in use a vacuum lock effect between the rigid cup and the inserted penis head for substantially locking the penis head to the cup to so that the penis head is forced to move along with the cup upon push/pull movement.

28. The device as claimed in claim 27, operable through any one or more of the following: i) to impart friction free pull/push reciprocation to the penis head; or ii) to transmit force through the penis and into the bulbospongiosus muscle or surrounding area for providing stimulation or activation of the penis, bulbospongiousus muscle or surrounding area; or to impart pull and push forces to act as a pump for blood circulation to force an increase in arterial blood circulation.

29. The device as claimed in claim 27, wherein the rigid cup and the actuator are configured to be detachable from one another.

30. The device as claimed in claim 27, wherein the rigid cup is configured to allow the penis head to be inserted and located in close-fitting relationship against a base end of the cup without air therebetween.

31. The device as claimed in claim 27, wherein the rigid cup has at least one air or fluid escape channel through a wall section of the rigid cup for letting out air or fluid from the cup when locating the penis head in the cup.

32. The device as claimed in claim 27, wherein the rigid cup or the coupling means includes the sealing means, and is arranged to extend around the penis.

33. The device as claimed in claim 27, wherein the coupling means comprises a sleeve which is either or both:
arranged to seal between the penis and the rigid cup for hindering or preventing air from entering the rigid cup; and
configured to grip the shaft of the penis at or near the penis head.

34. The device as claimed in claim 33, wherein the coupling means is movable between a first configuration in which the coupling means is supported on the rigid cup for permitting insertion of the penis head into the cup and a second configuration for coupling the cup to the penis, wherein in the second configuration, the coupling means is configured to seal between the penis and the rigid cup for hindering or preventing air from entering the rigid cup, and further configured to allow obtaining in use a vacuum lock effect between the penis head and the cup.

35. The device as claimed in claim 34, wherein the coupling means comprises a sleeve which is unrollable to move the coupling means from the first configuration to the second configuration, wherein in the second configuration the unrolled sleeve is configured to grip the penis.

36. The device as claimed in claim 33, wherein the rigid cup exterior has an outer wall portion between a base end and an entrance end, the outer wall portion comprising a circumferential groove or formation extending around the cup, and the sleeve is fitted to the rigid cup and a portion of the sleeve when fitted is disposed in or on the circumferential groove or formation.

37. The device as claimed in claim 27, further comprising a pump device for extracting air from a region between the penis head and the cup when the penis head is coupled to the cup.

38. The device as claimed in claim 27, further comprising control means for operating the actuator and/or a drive mechanism of the actuator.

39. The device as claimed in claim 38, wherein the control means comprises one or more user controllable buttons, switches or remote control.

40. The device as claimed in claim 27, wherein the rigid cup has a variable or adjustable rate of reciprocation.

41. The device as claimed in claim 27, wherein the rigid cup has a variable or adjustable travel length.

42. The device as claimed in claim 27, wherein the actuator is controllable to vary and/or adjust either or both the rate of reciprocation and travel length of the rigid cup.

43. The device as claimed in claim 27, wherein the actuator comprises a drive mechanism.

44. The device as claimed in claim 43, further comprising a hand-held casing from which a reciprocally movable drive part extends and is operable by the drive mechanism, wherein the drive mechanism is contained within the casing.

45. The device as claimed in claim 43, wherein the drive mechanism is configured to provide an adjustable travel length, and comprises:
a motor;
a rotary mover arranged to be driven and rotated by the motor;
a linear drive part arranged to be movable linearly back and forth;
an adjustable support member; and
a link member supported upon the adjustable support member via a rotational coupling;
a first coupling member arranged to extend between the link member and the rotary mover, the first coupling member having a first end which is coupled to the rotary mover and a second end which is coupled to the link member;
a second coupling member arranged to extend between the link member and the linear drive part, the second coupling member having a first end which is coupled to the link member and a second end which is coupled to the linear drive part; and
an adjuster for adjusting the position of the support member and control means for operating the adjuster, the control means being operable by a user by means of one or more user-operable control devices;
wherein the support member is positionally adjustable to move the rotational coupling between a first location and a second location, for varying the range of travel obtainable by the linear drive part when operating the rotary mover.

46. The device as claimed in claim 27, having a strap attached thereto, which strap is configured to extend around a neck of a user to support the device in position when in use or to allow for handsfree solution.
